(19) United States
(12) Patent Application Publication
(10) Pub. No.: US 2011/0035014 A1
(43) Pub. Date: Feb. 10, 2011

(54) HUMERAL COMPONENT

(75) Inventor: Michael Forrer, Seuzach (CH)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER GMBH, Winterthur (CH)

(21) Appl. No.: 12/161,284

(22) PCT Filed: Jan. 19, 2007
(86) PCT No.: PCT/EP04/50524

§ 371 Date: Aug. 8, 2008

(30) Foreign Application Priority Data
Jan. 20, 2006 (EP) 06100680.5
Apr. 27, 2006 (CH) 00698/06

(51) Int. Cl. A61F 2/40

(52) U.S. Cl. 623/19.14

(57) ABSTRACT

The invention relates to a humeral component of a shoulder joint prosthesis, comprising a bearing body (38), a shaft (32) and a transition area (34) which is arranged between the bearing body (38) and the shaft (32). One surface of the transition area (34) comprises, on a large area, a structure in the form of projections (48) for attaching bone fragments and/or muscle attachments and/at tendon attachments. The projections (48) are arranged discretely in a distributed manner on the surface of the transition area (34) and essentially in the proximal-distal direction and also essentially in the direction of the periphery.

The present invention relates to a humerus component of a shoulder joint prosthesis.

The use of shoulder joint prostheses can be necessary if the shoulder joint of a patient is damaged and therefore causes pain and/or if the functional capability of the joint is restricted.

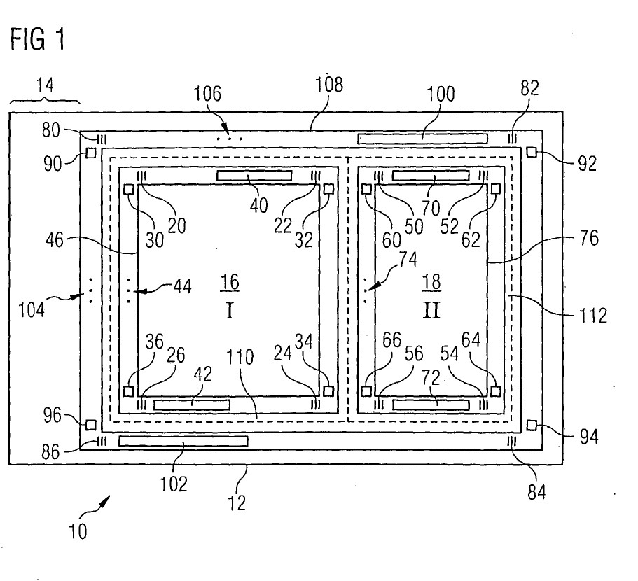

A right humerus 10 (long bone of the arm) is shown schematically in a view from the front in FIG. 1. It is divided into the humerus shaft 12 and a proximal end and a distal end. The distal end of the humerus 10 is substantially formed by the humerus condyle 14 (condylus humeri) and the medially and laterally arranged epicondyles 16. The roll-shaped humerus condyle 14 serves for the articulated connection to the ulna (not shown). The proximal end of the humerus 10 is substantially composed of a humerus head 18 (caput humeri), a humerus neck (collum anatomicum) and a major tubercle 22 (tuberculum majus) as well as a minor tubercle 24 (tuberculum minus). An intertubercular groove 26 (sulcus intertubercularis) is disposed between the two tubercles 22, 24.

The boney structure of the shoulder joint (glanohumeral joint) consist of the approximately spherical humerus head 18 and the joint socket of the shoulder blade (glenoid, not shown). The joint socket is comparatively shallow so that the contact surface to the oppositely disposed humerus head 18 is relatively small. Since no strongly pronounced ligaments are present, the muscles surrounding the joint have to stabilize it. A muscle-secured joint is spoken of in this case.

With degenerative joint diseases, for example due to abrasion (arthrosis) or inflammatory joint diseases (arthritis), a surface replacement may be indicated for the humerus head 18 in many cases. This is also indicated with humerus head necrosis. With advancing damage to the proximal humerus, it may be necessary under certain circumstances to make use of a primary total endoprosthesis.

As a consequence of accidents, fractures frequently also occur in the region of the proximal humerus 18 which cannot be treated by function-maintaining surgery. Depending on the severity of the injury, the supplying vessels of the humerus head 18 are frequently also destroyed so that the mortification of the humerus head 18 must be anticipated. There is the possibility in these cases of replacing the humerus head 18 by a prosthesis.

Fractures in the region of the proximal humerus are divided into different types in dependence on the number of arising fragments, on the extent of the fragment displacement and on the course of the fracture. “One-part fractures”, i.e. traumas with substantially only one fractured surface, without any displacement of the individual fragments with respect to one another, are called type 0 fractures and usually do not have to be treated surgically. Type A fractures have two fragments and are frequently characterized by the tearing off of the major tubercle 22 or of the minor tubercle 24. Fractures of the type B, C and X differ in the position of the fractured surfaces and usually have two to four bone fragments. With these fracture types and with a humerus head comminuted fracture, the proximal region of the humerus has to be stabilized by means of wires, screws or plates. In severe cases, the use of a shoulder joint prosthesis may also be indicated, as briefly mentioned above.

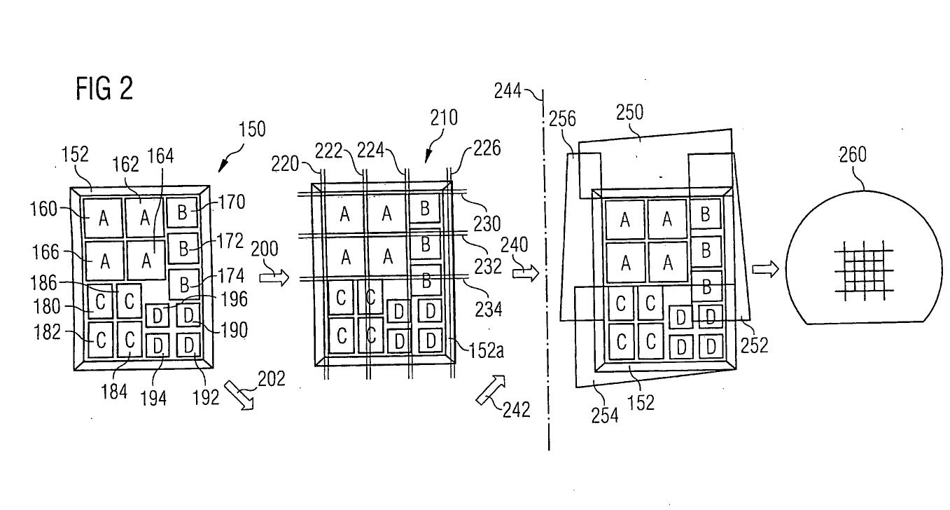

FIG. 2 illustrates, with reference to the dashed lines, typical fracture edges 28 such as occur with fractures in the region of the proximal humerus 10. Tears of the tubercles 22, 24 and/or fractures in the region of the humerus neck 20 and of the proximal humerus shaft occur frequently 12.

Prostheses for the restoration of the function of the shoulder joint are generally known.

EP 1415621 describes a shoulder joint prosthesis having two cooperating support bodies, a shaft and a coupling for the connection of the shaft to a support body on the humerus side. This system is very flexible and allows the choice of a support body suitable for the respective case. The support body can thus, for example, model the anatomy of the humerus head. In accordance with an alternative embodiment, however, the function of the spherical shell and the support shell can also be swapped over. In addition, with the known humerus head prosthesis, different positions of the support head can be realized relative to the shaft to take account of the individual anatomical demands of the different patients.

A shoulder endoprosthesis is furthermore known from EP 1093777 which has different elements for the fixing of bone fragments. These elements can, for example, be lugs, eyelets, claws and boreholes.

A prosthesis is known from EP 1 216 668 having a shaft which has a surface structure in the form of elongated ribs which extend substantially in the peripheral direction around an axis extending in the proximal-distal direction.

An innovative humerus components of a shoulder joint prosthesis should now be provided. The humerus component described in the following enables the reconstruction of the shoulder joint and thus a restoration of normal kinematics while largely maintaining the bone substance. In addition to a series of further properties, the humerus component can be fixed safely and reliably in the humerus shaft and furthermore allows the reliable fixing of bone fragments of the proximal humerus to the humerus component. A good anchorage of the support body replacing the humerus head can thus be ensured.

The humerus component of a shoulder joint prosthesis provided here thus includes a support body, a shaft and a transition region. The transition region is arranged between the support body and the shaft. The shaft of the humerus component is provided to be implanted in a humerus. The transition region of the humerus component, that is the region substantially including the metaphysis and parts of the epiphysis of the humerus, is provided with a structure in the form of elevated portions over a wide area, with the elevated portions being arranged discretely distributed over the surface of the transition region both substantially in the proximal-distal direction and substantially in the peripheral direction.

The elevated portions therefore do not form any elongated rib-like surface structures, but rather basically any desired discrete distribution over the surface, with a strict alignment of the elevated portions in the proximal-distal direction and—seen in a cross-section perpendicular to the proximal-distal direction—in the peripheral direction not being mandatory. It is rather the case that a “proximal-distal direction” and a “peripheral direction” are also to be understood as a random distribution of the elevated portions over the surface, that is as a discrete arrangement of the elevated portions in all directions. In other words, it is also possible in the present case to speak of spot tips or “spot-like” tips, in particular tips converging in substantially spot form, in contrast to rib-like and thus linear elevated portions.

This structure facilitates the fixing of bone fragments such as typically occur in humerus fractures, in particular when a plurality of fragments are present.

In addition, the structure of the attachment of muscles and ligaments to the prosthesis is supported. This is particularly of special importance with prostheses of the shoulder joint since, as already stated above, the stability of the shoulder joint substantially depends on the muscles surrounding the joint.

A high primary and long-term stability of the humerus component is thus therefore ensured.

Further embodiments of the invention are set forth in the dependent claims, in the description and in the drawings.

The humerus component can substantially completely have a structure in the form of elevated portions both at its anterior and at its posterior surface of the transition region. In addition, the lateral surface of the transition region can equally be provided with elevated portions. Such a humerus component takes the typical fracture geometries into account and enables the secure fixing of both the major tubercle and of the minor tubercle and other bone fragments.

In an embodiment, the transition region extends over at least 10% of the proximal-distal total length of the humerus component, in particular at least 15% and furthermore, in particular at least 20%. A very good purchase of the bone fragments and/or muscle/ligament attachments is ensured by provision of this proximal-distal extent of the transition region.

The transition region is in particular provided with the elevated portions completely over its total proximal-distal extent.

Provision can be made for the structure to be provided over a peripheral region of a cross-section perpendicular to the proximal-distal extent of the humerus component of at least 240° C. This peripheral region can in particular also include at least 270°. In other words, the structural elements forming the structure can be arranged in a strip-shaped or strip-like region which extends over a large part of the periphery. The primary stability of the reconstructed proximal humerus end is improved by the arrangement of elevated portions in such a large peripheral region. Such an embodiment of the transition region of the humerus component is of advantage particularly when a plurality of bone fragments are present which are to be fixed to the prosthesis from different sides.

In an embodiment, the transition region includes the metaphysary region of the humerus component which is arranged between the epiphysis and the diaphysis in the healthy joint or in the complete joint prosthesis.

In accordance with an embodiment of the humerus component, the elevated portions are shaped substantially regularly. These elevated portions can, for example, substantially have the shape of pyramids with a rectangular or square base and/or the shape of cones with a circular, oval or elliptical base. The elevated portions can furthermore be arranged distributed substantially regularly over the surface of the transition region.

With these measures, which can be realized individually or in combination, the surface is optimized with respect to its functional properties, that is the attachment of muscles and ligaments is facilitated and the fixing of bone fragments of complicated traumatic damage to the proximal humerus can be carried out simply and reliably. In addition, they ensure a cost-effective production of the prosthesis which nevertheless satisfies high quality demands.

Provision can furthermore be made for the support body and the transition region to be separably connected to one another. The ideal components for the specific case can be combined by this modular design. A large selection of shaft components, symmetrical and asymmetrical head and glenoid components is thus available and enables an ideal restoration of the anatomy of the shoulder joint. In addition, a modular system is easier to implant and to adapt.

Provision can additionally be made for a metaphysary base plate to be arranged between the transition region and the support body, which can be designed as a support sphere or a support shell, for example. This base plate can likewise have elevated portions for the attachment of bone fragments and/or muscle attachments and/or ligament attachments. The base plate can satisfy additional support functions and can promote the growing together of the bone fragments and muscle attachments or ligament attachments due to its substructure.

In a further embodiment, this metaphysary base plate has an outer support surface which at least partly fills a gap present between the bearing body and the shaft. This outer support shaft can likewise at least partly have a structure in the form of elevated portions. A substantially closed surface adjoining the lower side of the support body thus results by the support surface together with the surface of the transition region. Such a closed embodiment supports the growing together of the natural components of the affected body region. The anchorage of the humerus component is additionally improved by the elevated portions on the support surface.

The outer support surface of the metaphysary base plate can have a lateral groove. A lateral groove can also be arranged in a proximal region of the transition region. Such lateral grooves are provided for the fastening of tuberosities (bone projections with a rough surface to which the ligaments of muscles are attached). One or more bores, which can be arranged adjacent to and/or directly at the groove, facilitate the fastening of the tuberosities so that they can be arranged reliably and anatomically correctly beneath the humerus head or its prosthesis. Impingements and dislocations of the tubercles are thereby avoided. In this connection an “impingement” (“bottleneck”) designates the clamping of ligaments, cartilaginous joint lips or mucosal folds in the region of joints which results in a painful dysfunction. Impingement syndromes frequently affect the shoulder joint as an impingement syndrome of the rotator cuff, i.e. of the muscle group which holds the head of the humerus in the very shallow joint socket of the shoulder blade.

In accordance with an embodiment, the support body and the metaphysary base plate and/or the metaphysary base plate and the transition region are separably connected to one another. The basic advantages of the modular construction were already stated above. The modular base plate furthermore facilitates the replacement of the support body. Revision operations and adaptation operations can thus be carried out more simply and gently.

In a further embodiment, the transition region is made free of projecting/protruding fixing lugs or fixing eyelets. Such flanges or eyelets are expensive in production and moreover difficult to handle.

In accordance with an embodiment, the humerus component has bores in the transition region for the fixing of bone fragments with the help of taut threads or taut wires. In comparison with lugs or eyelets protruding from the surface of the transition region, bone fragments can be pressed better toward the transition region with the help of wires or threads by the use of bores. The fixing effect of the elevated portions is developed in an advantageous manner by this pressing. In other words, a larger pressing force of the bones fragments is thus achieved.

The invention furthermore relates to a shoulder joint prosthesis which includes a humerus component in accordance with at least one of the embodiments described above. Such a shoulder prosthesis is characterized by a high primary stability and allows a good reconstructions of the function of the glenohumeral joint. The prosthesis design, in particular the embodiment of the transition region of the humerus component, supports the growing together of bone fragments in the proximal region of the humerus.

All indications of alignment, positioning, orientation and direction which are used as required in the description and in the drawings in connection with the humerus component and in accordance with the technically usual convention and which in particular relate to anatomical axes, planes, directions in space and directions of movement are familiar to the person skilled in the art and relate to the implanted state of the humerus component.

The invention will be described in the following purely by way of example with reference to advantageous embodiments and to the drawings.

FIG. 1 shows a schematic representation of a right humerus in a view from anterior (see introduction);

FIG. 2 shows a schematic representation of the proximal end of a right humerus with typical break edges with traumatic damage in a view from anterior (see introduction);

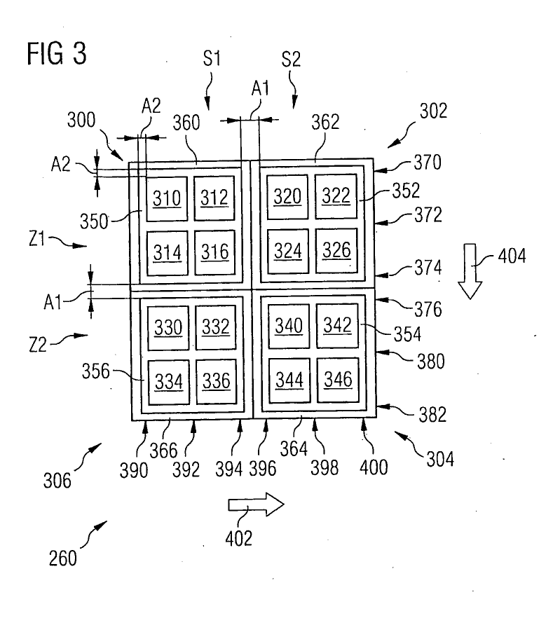

FIG. 3a shows a schematic representation of an embodiment of a proximal right humerus component in a perspective view from anterior;

FIG. 3b shows the embodiment of a proximal right humerus component shown in FIG. 3a in a perspective view obliquely from anterior;

FIG. 3c shows the embodiment of a proximal humerus component shown in FIG. 3a in a perspective view from lateral;

FIG. 3d shows the embodiment of a proximal humerus component shown in FIG. 3a in an exploded drawing;

FIG. 4 shows an enlarged representation of FIG. 3d;

FIG. 5 shows an enlarged representation of FIG. 3a;

FIG. 6 shows an enlarged representation of FIG. 3b;

FIG. 7 shows an enlarged representation of FIG. 3c;

FIG. 8 shows an embodiment of a proximal right humerus component in a perspective view obliquely from posterior in an exploded drawing;

FIG. 9 shows an embodiment of a proximal right humerus component in a view from anterior, with the support body being a support shell;

FIG. 10 shows the embodiment of a proximal humerus component shown in FIG. 9 in a view obliquely from anterior;

FIG. 11c shows the embodiment of a proximal humerus component shown in FIG. 9 in a view from lateral;

FIG. 12 shows a possible embodiment of an elevated portion of the structure.

FIGS. 3a to 3c are different views of embodiments of a humerus component 30. As can clearly be seen from FIG. 3d, the humerus component 30 is substantially divided into a shaft 32, a transition region 34, a metaphysary base plate 36 and a support head 38, with the shaft 32 and the transition region 34 forming a unit. These three individual assemblies of the humerus component 30 can be joined together and fixed by a clamping screw 40. A detailed description of the embodiment shown will be given with reference to the following Figures.

FIG. 4 is an enlarged view of FIG. 3d. The humerus shaft 32 is provided for implantation into a humerus 10. A longitudinal groove 42 can be seen which serves inter alia for the rotationally fixed fixation of the shaft 32 in a humerus 10. The shaft 32 has a slightly conical geometry)(1.5°) and increases in circumference in the course from distal to proximal. The transition region 34 adjoins the shaft 32 at proximal. The transition region 34 has a structured surface with pyramid-shaped elevated portions 48. The elevated portions 48 contribute to the improvement of the primary stability of the prosthesis in that they fix the bone fragments and promote the attachment of muscles. The surface is additionally rough-blasted.

In the embodiment shown here, the pyramid-shaped elevated portions 48 are uniformly distributed. In other embodiments, the distribution of the elevated portions 48 can also be irregular. For example, regions in which an increased strain on the bone fragments is to be expected can have an increased density of elevated portions. The elevated portions 48 can, in contrast to the embodiment shown here, also have different shapes, geometries and extents depending on their position. Conical elevated portions or elevated portions of different types, such as also truncated cones or truncated pyramids, are likewise conceivable.

The transition region 34 is moreover provided at the shaft with bores 50 for the fixing of bone fragments, for example torn off tubercles. This can take place, for example, with the help of wires or threads.

The elevated portions 48 are arranged on the anterior surface, the posterior surface and the lateral surface of the transition region 34. This embodiment does not have any elevated portions at medial. Such a distribution has proved to be suitable for the typically occurring fracture geometries and enables a very good fixing of the bone fragments. The medial section of the transition region 34 can also be provided with elevated portions 48 for cases with special considerations.

In other words, the structure extends in the form of elevated portions 48 over a large part of the peripheral region of the transition region 34 of the humerus component 30. Provision can be made not to provide the total transition region 34 with elevated portions 48 in its distal-proximal longitudinal extent, but rather, for example, to provide a strip-shaped region with elevated portions 48, for example, which extends over a large part of the transition region 34. Bone fragments can thereby be attached from practically all sides (lateral, medial, anterior and posterior).

In the embodiment shown, the elevated portions have the form of pyramids with a square base. The height of the elevated portions amounts to less than 2.5 mm, but can generally also amount to less than 2 mm. Embodiments with elevated portion heights of more than 0.5 mm and in particular more than 1 mm and, for example, 1.5 mm can be provided. The flank angle of the pyramids amounts to approximately 60° in the embodiment shown.

The transition region 34 moreover has suitable recesses at its proximal end to be able to establish a connection with the metaphysary base plate 36 or directly with a support head 38. A thread for the clamping screw 40 is moreover provided. The recesses and the thread are not visible from this view.

The transition region 34 can be connected to the metaphysary base plate 36 at proximal. The metaphysary base plate 36 has an installation surface 52 at its distal end which is complementary to the installation surface 54 at the proximal end of the transition region 34. At its proximal end, the metaphysary base plate 36 has a circular or elliptical plate section 56. The metaphysary base plate furthermore has two pins 58, 58′ which can be introduced into complementary cut-outs in the transition region 46 and the support head 38. The distal pin 58′ facing toward the transition region 34 can have a polygonal cross-section, for example.

The metaphysary base plate 36 is furthermore provided with a support surface 60 which likewise has elevated portions 48 at its surface. The support surface 60 is provided with a bore 50 and a lateral groove 62. The lateral groove 62 enables additional swedging connections and/or the attachment of bone fragments such as tuberosities. The bore 50 is arranged in direct proximity to the lateral groove 62 for the better fixing of the bone pieces attached in this region. The fixation can take place by means of wires/threads guided through the bore 50. A plurality of bores can also be provided adjacent to the lateral groove 62. In this embodiment, the surface of the support surface 60 has both a lateral component and an anterior and a posterior component.

However, embodiments can also be provided in which the support body is arranged directly at the transition region 34 (see also FIGS. 9 to 11).

The individual parts of the humerus component 30 are clamped to one another by means of the clamping screw 40 which has a metric thread in an embodiment.

The humerus component 30 described above is designed for anatomical reconstructions of 4-fragment fractures, with in particular the proximal part being optimized in such a manner to model the natural anatomy. Depending on the severity of the injury and in dependence on the anatomical circumstances of the patient and on the fracture geometry, the humerus component 30 can have a different design, with the position and shape of the elevated portions and also of the bores also being able to vary. Different versions of the humerus component 30 are provided for the left shoulder and for the right shoulder.

FIG. 5 shows an embodiment of the humerus component 30 in a view from anterior in an assembled state. A medial cut-out 63 can be recognized at the metaphysary base plate 36. It makes it possible to minimize any potentially necessary resection of still present bone and simultaneously to improve the anchorage of the prostheses in the bone.

FIG. 6 shows the humerus component 30 in a view obliquely from the front and illustrates the large-area arrangement of elevated portions 48 both on the transition region 34 and on the support surface 60 of the metaphysary base plate 36. It can moreover be seen from this Figure that the distal edges of the support head 38 are rounded in order not to damage the muscles surrounding the joint.

A lateral view of the humerus component 30 can be seen from FIG. 7. It can be seen that the shaft 32 has two grooves 42 in its lateral region to secure the rotational stability of the humerus component.

FIG. 8 shows a further representation of the humerus component 30. A set-back support installation surface 46 can be seen in this view. The support installation surface 46 forms a cut-out into which the plate section 56 of the metaphysary base plate 36 is lowered in the installation. The support head 38 additionally has an installation bore 59 for the reception of the pin 58.

FIG. 9 shows an inverse right humerus component 64. In this case, the support body is a concave articulation body, a so-called support shell 66. In the case shown, the support shell 66 is composed of an articulation element 68 and a fixing element 70. The complementary convex joint element of such an inverse shoulder prosthesis is fastened to the joint socket of the shoulder blade (not shown).

The support shell 66 is secured directly to the proximal end of the transition region 34. The unit of transition region 34 and shaft 32 is similar to the corresponding unit in FIG. 4. It can thereby be seen that this unit can serve so-to-say as a foundation for differently shaped metaphysary base plates 36 and/or support bodies 38, 66. Adaptations and revisions of the shoulder prosthesis can therefore be carried out easily.

FIGS. 10 and 11 show the inverse humerus component 64 shown in FIG. 9 from a view obliquely from anterior at the front or lateral.

FIG. 12 shows a possible embodiment of an elevated portion 48. A truncated pyramid is shown with an irregular base which has flanks with different flank angles. Irregular cones and/or truncated cones can generally also be provided. Elevated portions with different flank gradients can, for example, be arranged at positions at which mainly a directed strain is to be expected.

Depending on demands, the shape and the material of the humerus component 30, 64 can be optimized for cemented and uncemented applications.

### REFERENCE NUMERAL LIST

10 humerus
12 humerus shaft
14 humerus condyle
16 epicondyle
18 humerus head
20 humerus neck
22 major tubercle
24 minor tubercle
26 tubercle groove
28 fracture edges
30 humerus component
32 shaft
34 transition region
36 metaphysary base plate
38 support head
40 clamping screw
42 longitudinal groove
46 support installation surface
48 elevated portion
50 bore
52, 54 installation surface
56 plate section
58, 58′ pin
59 installation bore
60 support surface
62 lateral groove
63 medial cut-out
64 inverse humerus component
66 bearing shell
68 articulation element
70 fixing element

1-20. (canceled)

21. A humerus component of a shoulder joint prosthesis, comprising a support body, a shaft and a transition region arranged between the support body and the shaft, and also a metaphysary base plate which is arranged between the transition region and the support body, wherein a surface of the transition region has a structure over a large area in the form of discrete elevated portions for the attachment of bone fragments and/or of muscle attachments and/or of tendon attachments, and wherein the discrete elevated portions are arranged distributed discretely over the surface of the transition region both substantially in the proximal-distal direction and substantially in the peripheral direction, wherein the surface of the metaphysary base plate likewise has a structure in the form of elevated portions at least in part, with the elevated portions having the form of individual elements tapering to a point.

22. A humerus component in accordance with claim 21, wherein an anterior surface and a posterior surface of the transition region as well as a lateral surface of the transition region substantially completely have a structure in the form of elevated portions.

23. A humerus component in accordance with claim 21, wherein the proximal-distal extent of the transition region comprises at least 15% of the proximal-distal total length of the humerus component, in particular at least 20% and furthermore in particular at least 25%.

24. A humerus component in accordance with claim 21, wherein the structure is arranged on a peripheral region of a cross-section perpendicular to the proximal-distal extent of the humerus component of at least 240°, in particular at least 270°.

25. A humerus component in accordance with claim 21, wherein the elevated portions are arranged distributed in a substantially regular manner over the surface of the transition region.

26. A humerus component in accordance with claim 21, wherein the elevated portions substantially have the form of pyramids and/or cones and/or of truncated pyramids and/or truncated cones.

27. A humerus component in accordance with claim 21, wherein the height of the elevated portions amounts to less than 2.5 mm, in particular to less than 2 mm.

28. A humerus component in accordance with claim 21, wherein the height of the elevated portions amounts to more than 0.5 mm, in particular to more than 1 mm and furthermore in particular to more than 1.5 mm.

29. A humerus component in accordance with claim 21, wherein the metaphysary base plate has a support surface which fills a gap present between the support body and the shaft at least in part such that the growing together of bone fragments is promoted, with the support surface likewise having a structure in the form of elevated portions at least in part.

30. A humerus component in accordance with claim 21, wherein the support surface of the metaphysary base plate has at least one bore.

31. A humerus component in accordance with claim 21, wherein the support surface of the metaphysary base plate has at least one lateral groove.

32. A humerus component in accordance with claim 31, wherein the bore is arranged in direct proximity to the lateral groove.

33. A humerus component in accordance with claim 21, wherein the support body and the metaphysary base plate and/or the metaphysary base plate and the transition region are separably connected to one another.

34. A humerus component in accordance with any claim 21, wherein the transition region is free of projecting fixing lugs or fixing eyelets.

35. A shoulder joint prosthesis comprising a humerus component which is made in accordance with claim 21.
